(19) United States
(12) Patent Application Publication
(10) Pub. No.: US 2023/0334678 A1
(43) Pub. Date: Oct. 19, 2023

(54) OPHTHALMIC IMAGE REGISTRATION USING INTERPRETABLE ARTIFICIAL INTELLIGENCE BASED ON DEEP LEARNING

(71) Applicant: Alcon Inc., Fribourg (CH)

(72) Inventors: Lu Yin, Keller, TX (US); Ramesh Sarangapani, Coppell, TX (US); Vignesh Suresh, Woodland Hills, CA (US)

(21) Appl. No.: 18/299,029

(22) Filed: Apr. 11, 2023

Related U.S. Application Data
(60) Provisional application No. 63/332,135, filed on Apr. 18, 2022.

(51) Int. Cl. G06T 7/33; G06T 7/11; G06T 7/00; G06T 7/37; A61B 3/00

(52) U.S. Cl. CPC G06T 7/337; G06T 7/11; G06T 7/0014; G06T 7/37; A61B 3/0025; G06T 2207/20084; G06T 2207/10101; G06T 2207/30041; G06T 2207/10012; G06T 2207/20081

(57) ABSTRACT

In certain embodiments, an ophthalmic system and computer-implemented method for performing ophthalmic image registration are described. The ophthalmic image registration includes obtaining a plurality of images of an eye of a user. Within each image of the plurality of images, a segmented region(s) of the eye within the image is determined based on evaluating the image with a neural network(s), and a set of point features of the eye within the segmented region(s) of the eye is determined based on evaluating the image with the neural network(s). A set of transformation information for transforming at least one of the plurality of images is generated based on performing one or more image processing operations on the set of point features within each image of the plurality of images. At least one of the plurality of images is transformed, based on the set of transformation information.

### BACKGROUND

Image registration is generally the process of transforming different image datasets into one coordinate system with matched imaging contents (or features). Image registration may be used when analyzing multiple images that were acquired from different viewpoints (or angles), acquired at different times, acquired using different sensors/modalities, or a combination thereof. The establishment of image correspondence through image registration is crucial to many clinical tasks, including but not limited to, ophthalmic microsurgical procedures (e.g., vitreoretinal procedures, such as retinotomies, retinectomies, autologous retinal transplants, etc., anterior segment surgery procedures, such as cataract surgery, minimally invasive glaucoma surgery (MIGS), etc.), diagnostic monitoring and evaluation (e.g., disease diagnosis and/or stage evaluation), treatment, etc.

### SUMMARY

In certain embodiments, an ophthalmic system for performing ophthalmic image registration is provided. The ophthalmic system includes one or more ophthalmic imaging devices, a memory including executable instructions, and a processor in data communication with the memory. The one or more ophthalmic imaging devices are configured to generate a plurality of images of an eye of a user. Each of the plurality of images includes a different view of the eye. The processor is configured to execute the executable instructions to determine, within each image of the plurality of images, one or more segmented regions of the eye within the image, based on evaluating the image with one or more neural networks. The processor is also configured to execute the executable instructions to determine, within each image of the plurality of images, a set of point features of the eye within the one or more segmented regions of the eye, based on evaluating the image with at least one of the one or more neural networks. The processor is further configured to execute the executable instructions to generate a set of transformation information for transforming at least one of the plurality of images, based on performing one or more image processing operations on the set of point features within each image of the plurality of images. The processor is further configured to execute the executable instructions to transform the at least one of the plurality of images, based on the set of transformation information. The processor is configured to transform the at least one of the plurality of images by at least one of scaling the at least one of the plurality of images, translating the at least one of the plurality of images, or rotating the at least one of the plurality of images, such that the plurality of images with the different views are in a same coordinate system.

In certain embodiments, a computer-implemented method for performing ophthalmic image registration is provided. The computer-implemented method includes obtaining a plurality of images of an eye of a user. Each of the plurality of images includes a different view of the eye. The computer-implemented method also includes determining, within each image of the plurality of images, one or more segmented regions of the eye within the image, based on evaluating the image with one or more neural networks. The computer-implemented method also includes determining, within each image of the plurality of images, a set of point features of the eye within the one or more segmented regions of the eye, based on evaluating the image with at least one of the one or more neural networks. The computer-implemented method further includes generating a set of transformation information for transforming at least one of the plurality of images, based on performing one or more image processing operations on the set of point features within each image of the plurality of images. The computer-implemented method further yet includes transforming the at least one of the plurality of images, based on the set of transformation information. Transforming the at least one of the plurality of images includes at least one of scaling the at least one of the plurality of images, translating the at least one of the plurality of images, or rotating the at least one of the plurality of images, such that the plurality of images with the different views are in a same coordinate system.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1A illustrates an example system for performing ophthalmic image registration, according to certain embodiments.

FIG. 1B illustrates another example system for performing ophthalmic image registration, according to certain embodiments.

FIG. 1C illustrates another example system for performing ophthalmic image registration, according to certain embodiments.

FIG. 2 illustrates an example workflow for performing ophthalmic image registration, according to certain embodiments.

FIG. 3 illustrates another example workflow for performing ophthalmic image registration, according to certain embodiments.

FIG. 4 illustrates an example architecture of a neural network for performing ophthalmic image registration, according to certain embodiments.

FIG. 5 is a flowchart of an example method for performing ophthalmic image registration, according to certain embodiments.

FIG. 6 is a flowchart of an example method for performing ophthalmic image registration, according to certain embodiments.

FIG. 7 illustrates an example workflow of a first stage of an ophthalmic image registration procedure, according to certain embodiments.

FIG. 8 illustrates another example workflow of a first stage of an ophthalmic image registration procedure, according to certain embodiments.

FIG. 9 illustrates an example workflow of a second stage of an ophthalmic image registration procedure, according to certain embodiments.

FIG. 10 illustrates another example workflow of a second stage of an ophthalmic image registration procedure, according to certain embodiments.

FIG. 11A illustrates example set of input images for ophthalmic image registration, according to certain embodiments.

FIG. 11B illustrates an example of a transformed image after performing ophthalmic image registration using the set of input images illustrated in FIG. 11A.

FIG. 11C illustrates an example image overlay with the images illustrated in FIG. 11B.

FIG. 12 illustrates an example computing system for performing ophthalmic image registration, according to certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Conventional image registration generally involves iteratively performing a number of tasks, including, for example, feature extraction, feature matching, transform model estimation, and image resampling and transformation in order to find an optimal alignment between images. One issue with conventional image registration is that iteratively performing these tasks can be resource inefficient (e.g., time/compute consuming and process-intensive process), making it unsuitable for many time critical and/or resource-limited applications. Another issue with conventional image registration is that it can lead to inaccurate results, since the process generally converges to local optima (e.g., a solution that is optimal within a neighboring set of candidate solutions) as opposed to the global optima (e.g., a solution that is optimal among all possible solutions).

In some cases, deep learning approaches can be applied to image registration to increase the speed and accuracy of the registration process. In one reference example, a deep learning based one-step transformation estimation technique can receive a pair of images to be registered as input, and then output a transformation matrix. However, one challenge with these deep learning-based registration approaches is that it can be difficult to procure and generate ground truth data. For example, in the ophthalmology domain, such ground truth data can include a reference standard of measurements and/or locations of various eye tissues/structures. Additionally, these deep learning approaches are generally non-interpretable due to the steps of feature extraction and feature matching being skipped. That is, due to the complexity of deep neural networks, what is learned in the hidden layers of the deep neural networks is generally unknown. This lack of interpretability generally makes it challenging to understand the behavior and prediction of deep learning neural networks. Accordingly, it may be desirable to provide improved deep learning based approaches for performing ophthalmic image registration.

Embodiments described herein provide systems and techniques for performing ophthalmic image registration using an interpretable deep learning based AI algorithm. An exemplary ophthalmic image registration technique described herein may use a deep neural network to detect point features (also referred to as key points) in one or more areas of the eye. The point features may correspond to unique features of the eye that may be persistent between different images of the eye. Collectively, the point features can be used as a fingerprint of the eye. After detecting the point features, the image registration technique may perform point matching and transform model estimation. In this manner, embodiments described herein can align images using an image registration technique that is interpretable (e.g., based on the point features), while achieving the speed and accuracy associated with deep learning based approaches (e.g., deep learning based one-step transformation estimation).

Additionally or alternatively, in certain embodiments, the ophthalmic image registration technique described herein can determine whether images are not able to be registered. For example, the ophthalmic image registration technique can receive images to be registered as input, determine a set of features within each image, determine, based on an analysis of the set of features from each of the images, a confidence score indicating a likelihood of successful registration of the images, and provide (e.g., display) an indication of the confidence score. If the confidence score is less than a threshold, then the ophthalmic image registration technique may determine that the set of images are not eligible for registration (e.g., there is a lack of correspondence between the image features). On the other hand, if the confidence score is greater than or equal to a threshold, then the ophthalmic image registration technique may determine that the set of images are eligible for registration (e.g., there is a correspondence between the image features).

While many of the following embodiments use a “U-NET” as a reference example of a deep learning artificial neural network that can be used by the ophthalmic image registration technique described herein, embodiments are not limited to the “U-NET” architecture and can include other types of deep learning artificial neural networks, such as an encoder-decoder network or auto-encoder. As used herein, a hyphenated form of a reference numeral refers to a specific instance of an element and the un-hyphenated form of the reference numeral refers to the collective element. Thus, for example, device “12-1” refers to an instance of a device class, which may be referred to collectively as devices “12” and any one of which may be referred to generically as a device “12”.

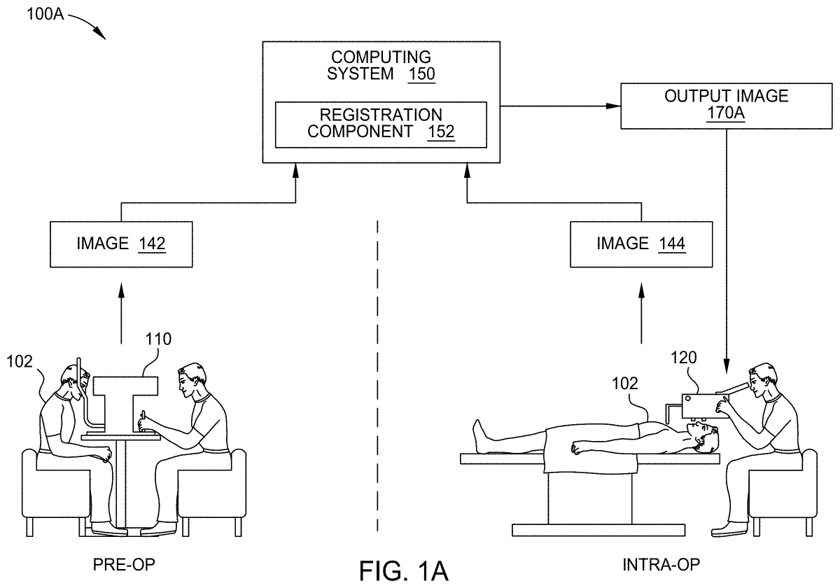

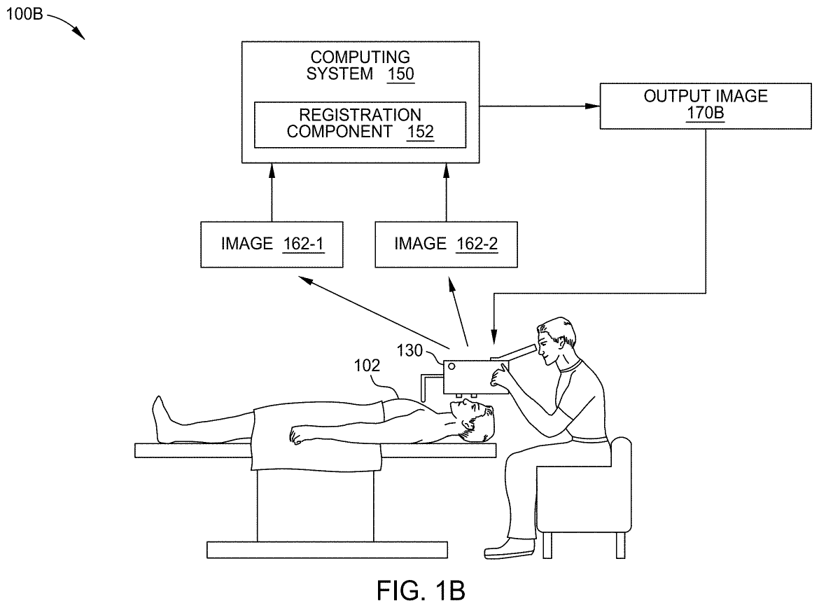

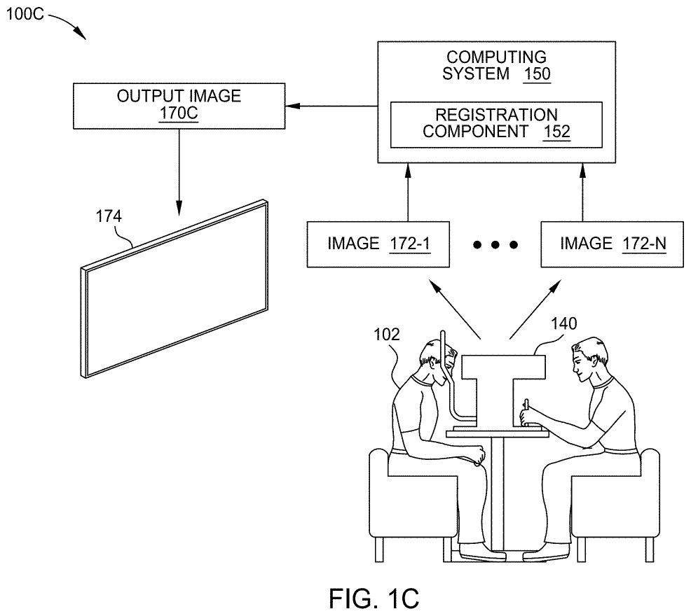

FIG. 1A illustrates a system 100A for performing ophthalmic image registration, according to certain embodiments. The system 100A includes an imaging device 110, a computing system 150, and an imaging device 120. The imaging devices 110, 120 are representative of a variety of imaging devices. More particularly, imaging device 110 is representative of a pre-operative (pre-op) imaging device that is used in a clinic to generate imaging of the patient's eye prior to surgery. An example of imaging device 110 may be an optical coherence tomography (OCT) device, an optical biometer, a digital keratometer, a rotating (e.g., Scheimpflug) camera, an MRI device, a digital microscope (e.g., a three-dimensional stereoscopic digital microscope), digital camera, digital fundus camera, and other imaging devices, now known or later developed.

Imaging device 120 is representative of an intra-operative (intra-op) that is used during a surgical procedure in the operating room to generating imaging of the patient's eye. An example of imaging device 110 is a digital microscope (e.g., a three-dimensional stereoscopic digital microscope), a digital camera, a digital fundus camera, and other imaging devices, now known or later developed. The computing system 150 is representative of a variety of computing systems (or devices), including, for example, a laptop computer, mobile computer (e.g., a tablet or a smartphone), a server computer, a desktop computer, an imaging system, a system embedded in a medical device, etc.

In certain embodiments, the system 100A performs image registration of multiple images 142, 144 captured of the patient's eye and presents an output of the image registration (e.g., output image 170A) to a user, such as a surgeon, during surgery. The images 142, 144 may include different views of the eye. The different views within the images 142, 144 may be due, in part, to the images 142, 144 being captured at different times, at different viewpoints, using different modalities, etc. Here, for example, the imaging device 110 can capture an image 142 of the eye of the patient 102 during the pre-op phase and the imaging device 120 can capture an image 144 of the eye of the patient 102 during the intra-op phase. In another example, the images 142, 144 may be captured during cyclorotation of the eye as the patient 102 changes from a sitting position to a supine position during surgery.

In certain embodiments, the image 142 includes one or more annotations (or markings) made by a user, such as a surgeon or surgical staff member. For example, during the pre-op phase, the user may assess clinical properties of the eye, based on the image 142, and annotate the image 142 with information associated with the clinical properties.

The computing system 150 includes a registration component 152, which is configured to implement one or more techniques described herein for performing ophthalmic image registration. The registration component 152 can include software components, hardware components, or a combination thereof. In certain embodiments, the registration component 152 obtains the images 142, 144, performs image registration on the images 142, 144, and generates an output image 170A, based on the image registration. In certain embodiments, the output image 170A is an image overlay of an aligned (or transformed) image 142 with image 144 (or an image overlay of an aligned (or transformed) image 144 with image 142). For example, the registration component 152 can generate the output image 170A in part by transforming the image 142 into the coordinate system of the image 144 or by transforming the image 144 into the coordinate system of the image 142.

As described in greater detail below, the registration component 152 can use an interpretable AI algorithm to perform ophthalmic image registration. In certain embodiments, the AI algorithm includes a first stage and a second stage. In the first stage, a deep learning neural network(s) (also referred to herein as a deep learning artificial neural network, an artificial neural network, or neural network) performs a segmentation operation on each image 142, 144. For example, the deep learning neural network(s) can transform each image 142, 144 into a different score map/segmentation map, where values in each score map/segmentation map indicates particular regions (or segments) of the eye (e.g., skin region, sclera, iris, pupil).

The second stage performs point feature detection (i.e., key point detection) in the eye for point matching. For example, the second stage can detect point features (corresponding to unique features) in the score maps/segmentation maps associated with the images 142, 144. Additionally, in the second stage, the detected point features in the score maps of the image 142 and the image 144 are matched using one or more computer vision algorithms (or techniques). As a reference example, a root scale-invariant feature transform (SIFT) computer vision algorithm can be used to detect and match the point features in the score maps of the images 142, 144. Additionally, a transformation matrix is generated (or computed) based on the matching points. The transformation matrix can be used to transform (e.g., scale, translate, rotate, etc.) the image 142 and/or the image 144 into a same coordinate system, such that output image 170A includes an alignment of the images 142 and 144.

Additionally, in certain embodiments, a set of features of the image 142 and a set of features of the image 144 may be extracted from one or more layers of the deep learning neural network(s) in the first stage. In the second stage, a classification component (also referred to as a classifier) can compare the set of features of the image 142 and the set of features of the image 144 to determine whether the images 142, 144 have a sufficient amount of features (e.g., above a threshold) available for registration. The classification component can generate a confidence score indicating a likelihood of success of the image registration, based on the comparison. The classification component can provide (e.g., display) an indication of the confidence score to a user (e.g., surgeon).

As shown in FIG. 1A, the output image 170A is provided to the imaging device 120, which can present the output image 170A for display to the user (e.g., surgeon). The display of the output image 170A may provide the user with the pre-op information regarding the eye as the user is viewing the eye via the imaging device 120, thereby improving the safety and/or effectiveness of a surgical procedure. For example, the output image 170A can be used for eye tracking during the surgical procedure, lens alignment during the surgical procedure, image guidance, robotic assisted surgery, etc.

Note that FIG. 1A illustrates a reference example of a system for performing ophthalmic image registration and that, in other embodiments, the system may have different configurations. For example, while FIG. 1A depicts the computing system 150 as separate from the imaging device 120, in some embodiments, the computing system 150 may be a part of the imaging device 120. Further, while FIG. 1A illustrates performing ophthalmic image registration for pre-op and intra-op images, in other embodiments, the techniques described herein can be used to perform ophthalmic image registration in other scenarios/applications. By way of example and not by way of limitation, FIG. 1B illustrates an example system 100B for performing ophthalmic image registration and FIG. 1C illustrates another example system 100C for performing ophthalmic image registration, according to certain embodiments.

The system 100B in FIG. 1B includes the computing system 150 and an imaging device 130. In certain embodiments, the system 100B in FIG. 1B can perform ophthalmic image registration for accurate and robust three-dimensional (3D) eye tracking. For example, the imaging device 130 may include a 3D sensor configured to capture images 162-1 and 162-2 of the eye of the patient 102. Assuming the 3D sensor is a stereo camera with two imaging sensors (or cameras or imagers) separated by a baseline, the image 162-1 may be captured with a first imaging sensor of the stereo camera and the image 162-2 may be captured with a second imaging sensor of the stereo camera. The computing system 150 can perform (via the registration component 152) an ophthalmic image registration of the images 162 1-2 to generate an output image 170B and can provide the output image 170B to the imaging device 130 for display (or presentation) to the user. The output image 170B can be used to diagnose various eye diseases, such as glaucoma, diabetic retinopathy, age-related macular degeneration, etc.

In certain embodiments, the system 100B in FIG. 1B can perform ophthalmic image registration for real-time tracking of certain eye tissues/structures (e.g., retinal vessels, sclera and limbus vessels, etc.). For example, the imaging device 130 may capture multiple images 162, where each image 162 corresponds to a different frame at a different instance in time. For instance, image 162-1 may correspond to a first frame at to, image 162-2 may correspond to a second frame at ti, and so on. In these embodiments, the computing system 150 (via the registration component 152) may generate multiple output images 170B over a period of time for real-time tracking, based on registration performed on the captured images 162.

The system 100C in FIG. 1C includes the computing system 150 and an imaging device 140. In certain embodiments, the system 100C can perform ophthalmic image registration for diagnostic evaluation and/or monitoring of a patent's eye. Here, for example, the imaging device 140 can capture multiple images 172 1-N of the eye of the patient 102. Each image 172 may be an image of a different portion of a particular eye tissue/structure (e.g., eye fundus). The computing system 150 (via the registration component 152) can perform ophthalmic image registration of the images 172 1-N to mosaic the images 172 1-N into a single image 170C. The computing system 150 can provide the image 170C to a display 174 and/or to the imaging device 140 for presentation to a user.

Note that while FIG. 1B depicts the computing system 150 as separate from the imaging device 130, in some embodiments, the computing system 150 may be a part of the imaging device 130. Similarly, while FIG. 1C depicts the computing system 150 as separate from the imaging device 140, in some embodiments, the computing system 150 may be a part of the imaging device 140.

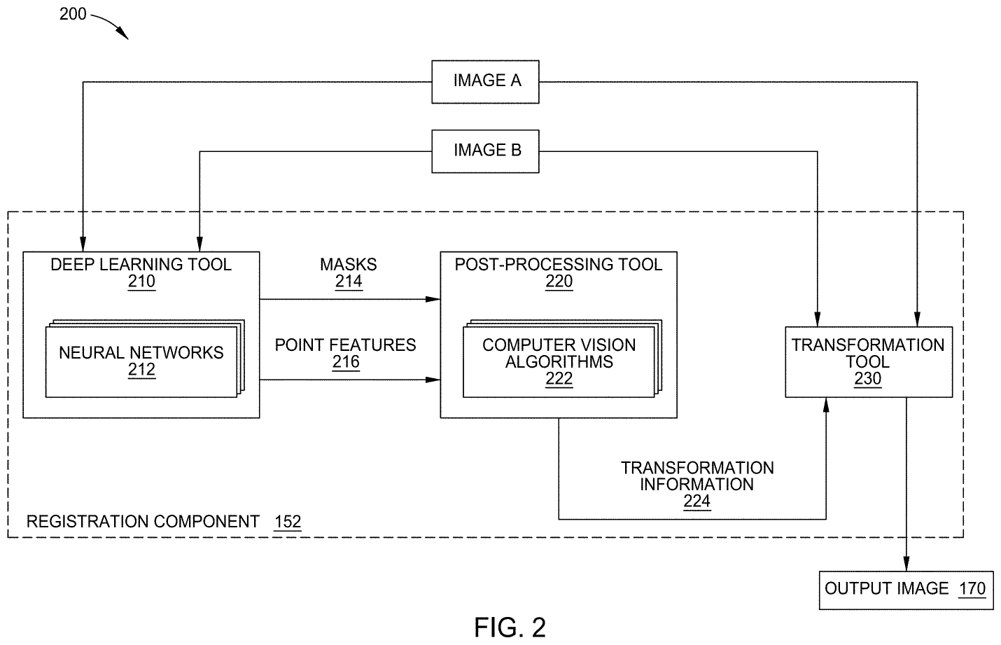

FIG. 2 illustrates an example workflow 200 for performing ophthalmic image registration, according to certain embodiments. The workflow 200 may be performed by the registration component 152. Here, the registration component 152 includes a deep learning tool 210, a post-processing tool 220, and a transformation tool 230, each of which can include hardware components, software components, or a combination thereof.

The deep learning tool 210 is generally configured to perform a segmentation operation on the input images A and B and to detect one or more point features within the input images A and B, using one or more neural networks 212 (also referred to as artificial neural networks, deep neural networks, etc.). The input images A and B may be representative of any set of input images waiting to be registered (e.g., images 142, images 162, images 172, etc.). The neural network(s) 212 is generally a deep learning network(s) which can be trained to perform various computer vision tasks, including, for example, image classification, image classification and localization, object detection, semantic segmentation, instance segmentation, etc.

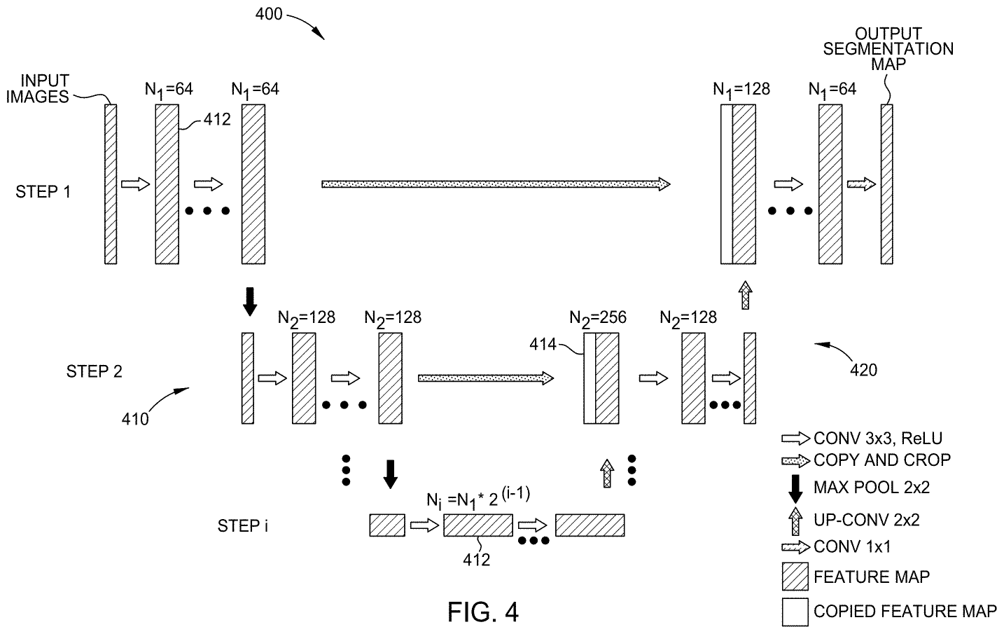

The neural network(s) 212 can include convolutional neural networks. In certain embodiments, the neural network(s) 212 is a U-net. Referring briefly to FIG. 4, FIG. 4 illustrates an exemplary U-net architecture 400, according to certain embodiments. The U-net architecture 400 includes (i) a number of multi-channel feature maps 412, each having a number of channels (N) and a particular x-y size, and (ii) a number of copied multi-channel feature maps 414. The U-net architecture 400 includes a contracting path 410 (left side) and an expansive path 420 (right side). The contracting path 410 may generally follow architecture of a convolutional network. For example, each step of the contracting path 410 may include repeated application of one or more (e.g., two) 3×3 convolutions (e.g., unpadded convolutions), followed by a rectified linear unit (ReLU) and a 2×2 max pooling operation with stride 2 for downsampling. At each downsampling step, the number of channels in each feature map 412 is doubled, relative to the previous step.

Each step in the expansive path 420 includes an upsampling of the feature map 412, followed by a 2×2 convolution (e.g., up-convolution) that halves the number of feature channels, a concatenation with the correspondingly cropped feature map 412 from the contracting path 410, and one or more (e.g., two) 3×3 convolutions, each followed by a ReLU. At the final layer, a 1×1 convolution is used to map the feature vector to a desired number of classes.

Referring back to FIG. 2, the deep learning tool 210 is configured to generate mask(s) 214 (also referred to as a segmentation result) and a set of point features 216 for each image A and image B, using the neural network(s) 212. The mask(s) 214 indicate different regions of the eye within the respective image. For example, the mask(s) 214 can indicate regions of the skin, sclera, iris, pupil, etc. The point features 216 generally include a set of feature within the respective image that can be used to uniquely identify the image. In some examples, the unique features may correspond to different blood vessels of the eye that are visible within the respective image.

In certain embodiments, the deep learning tool 210 can use a single neural network 212 to generate the mask(s) 214 and point features 216 for each image A and image B. In these embodiments, the neural network 212 may be trained on a dataset that includes a number of images, a set of point features within each image, and the boundaries of critical regions (e.g., sclera, iris, pupil, etc.) within each image. The point features and region boundaries within the set of training images may be annotated by a user, using machine learning tools, or a combination thereof. The single neural network 212 may be trained using a variety of algorithms, including, for example, stochastic gradient descent.

In certain embodiments, the deep learning tool 210 can use multiple neural networks 212 to generate the mask(s) 214 and the point features 216. For example, the deep learning tool 210 can use a first neural network 212 to generate the mask(s) 214 and use a second neural network 22 to generate the point features 216 for each image A and image B. In these embodiments, the first neural network may be trained on a dataset that includes a number of images and marked boundaries of critical regions within each image. The second neural network may be trained on a dataset that includes marked boundaries of critical regions within each image and a set of point features within the boundaries of the critical regions within each image. Each neural network 212 may be trained using a variety of algorithms, including, for example, stochastic gradient descent.

In FIG. 2, the post-processing tool 220 evaluates the mask(s) 214 and the point features 216 for the images A and B using one or more (or a combination of) computer vision algorithms 222 (or image processing operations) to generate a set of transformation information 224. The computer vision algorithms 222 can include, but are not limited to, SIFT, random sample consensus (RANSAC), thresholding, non-maximum suppression (NMS), etc. The transformation information 224 can include scaling information, translation information, and/or rotation information for transforming at least one of the images A and B, such that the point features 216 in each image A and B are aligned (or are in the same coordinate system). In certain embodiments, the transformation information 224 includes a homography matrix.

The transformation tool 230 receives the transformation information 224 and applies the transformation information 224 to at least one of the images A and B to generate an output image 170. As noted, in certain embodiments, the output image 170 may include an overlay of the images A and B aligned using the transformation information 224.

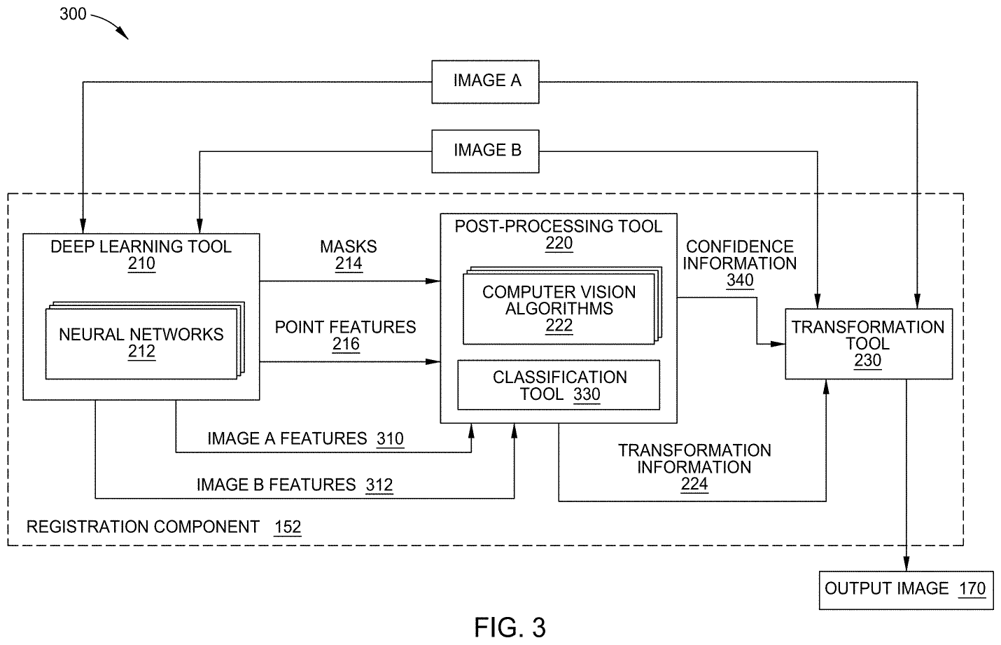

FIG. 3 illustrates an example workflow 300 for performing ophthalmic image registration, according to certain embodiments. The workflow 300 may be performed by the registration component 152. The workflow 300 is similar to the workflow 200, except that, in the workflow 300, the post-processing tool 220 also includes a classification tool 330, which can include software components, hardware components, or a combination thereof.

The classification tool 330 is generally configured to determine whether a set of input images (e.g., images A and B) are eligible for registration, based on an evaluation of a set of features from each input image. The classification tool 330 can generate a set of confidence information, based on the evaluation, indicating a likelihood that the transformation information 224 will result in a successful alignment of the set of images. Here, for example, the post-processing tool 220 (via the classification tool 330) receives a set of image A features 310 and a set of image B features 312 from the deep learning tool 210. In certain embodiments, the image A features 310 and the image B features 312 may be extracted from the middle layer of the neural network 212 (e.g., feature map 412 in step i of the U-net architecture 400 illustrated in FIG. 4).

The post-processing tool 220 (via the classification tool 330) may compare the image A features 310 with the image B features 312 and generate a set of confidence information 340, based on comparison. For example, the post-processing tool 220 can perform a cross-correlation of the image A features 310 with the image B features 312, and generate the confidence information 340, which includes an output (or result) of the cross-correlation. In certain embodiments, the transformation tool 230 may proceed to transform the images A, B using the transformation information 224, when the confidence information 340 indicates an amount of correlation between the images A features 310 and the image B features 312 is greater than or equal to a threshold.

On the other hand, in certain embodiments, the transformation tool 230 may refrain from transforming the images A, B using the transformation information 224, when the confidence information 340 indicates an amount of correlation between the image A features 310 and the image B features 312 is below a threshold. In these embodiments, the registration component 152 may generate feedback with an indication that the images A, B are ineligible for registration. In some embodiments, this feedback information may be used to update the training of the neural network(s) 212.

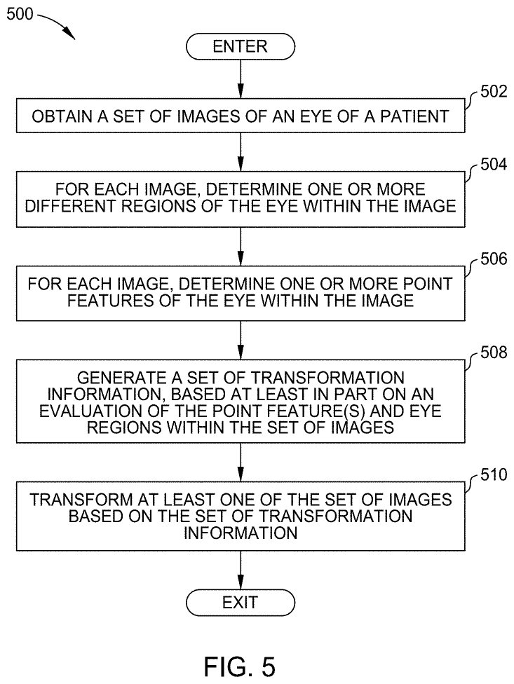

FIG. 5 is a flowchart of a method 500 for performing ophthalmic image registration, according to certain embodiments. The method 500 may be performed by a registration component (e.g., registration component 152).

Method 500 enters at block 502, where the registration component obtains a set of images of an eye of a patient (e.g., patient 102). Each image of the set of images may be captured at a different time, at a different angle/position, using a different sensor/modality, etc.

At block 504, the registration component, for each image, determines one or more different regions of the eye (e.g., masks(s) 214) within the image. For example, the registration component can perform a segmentation operation on each image using one or more neural networks (e.g., neural networks 212) to identify regions of the skin, sclera, iris, pupil, etc.

At block 506, the registration component, for each image, determines one or more point features (e.g., point features 216) of the eye within the image. The one or more point features may correspond to one or more unique landmarks or points of the eye. The registration component can generate a set of point features based on evaluating each image using the one or more neural networks (e.g., neural networks 212).

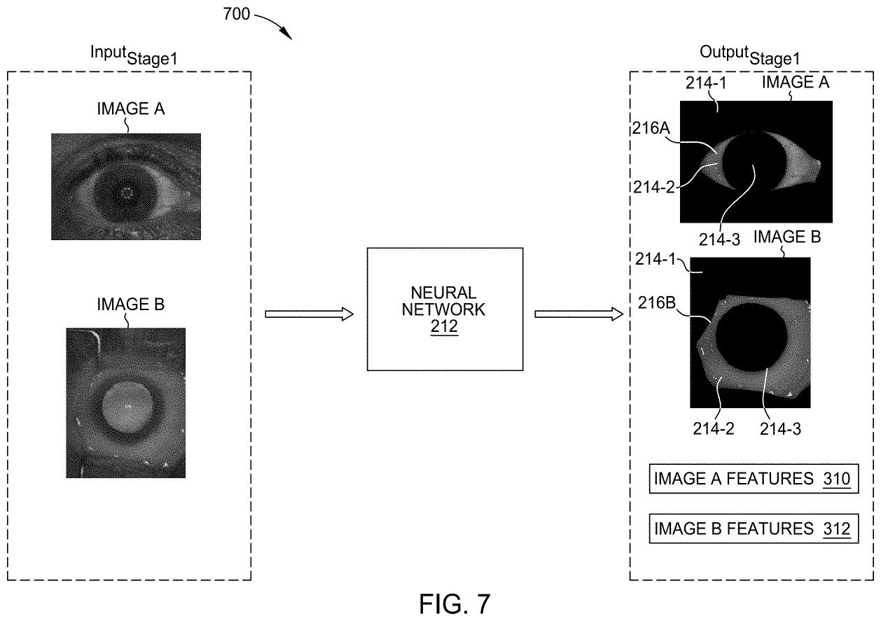

In certain embodiments, the operations in block 504 and block 506 may be performed using a single neural network 212. By way of example, FIG. 7 illustrates a first stage 700 of ophthalmic image registration using a single neural network 212 to identify the masks 214 and point features 216 within each input image A and B, according to certain embodiments. In the first stage 700, the neural network 212 can output, for each image, a mask 214-1 indicating a region of skin, a mask 214-2 indicating the sclera, and a mask 214-3 indicating the pupil. The neural network 212 can also output, for each image, a set of point features within the image (e.g., point features 216A for image A and point features 216B for image B).

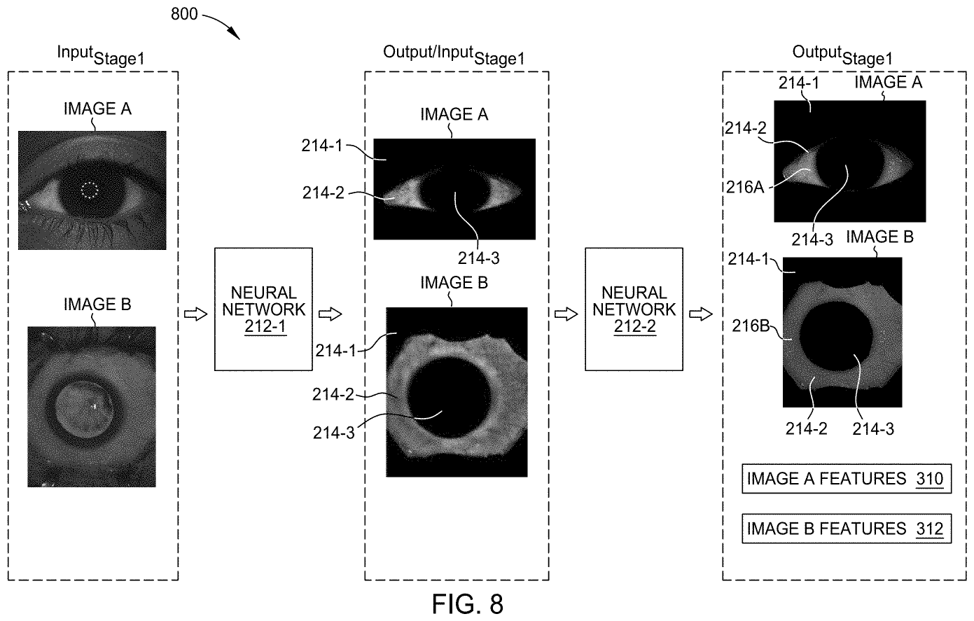

In certain embodiments, the operations in block 504 and block 506 may be performed using multiple neural networks 212. By way of example, FIG. 8 illustrates a first stage 800 of ophthalmic image registration using multiple neural networks 212-1 and 212-2, according to certain embodiments. In the first stage 800, the neural network 212-1 can segment different regions of each input image A and B. For example, the neural network 212-1 can identify, within each input image, a mask 214-1 indicating a region of skin, a mask 214-2 indicating the sclera, and a mask 214-3 indicating the pupil. The neural network 212-2 receives the mask (or segment) information output from the neural network 212-1 and generates, for each image, a set of point features within the image (e.g., point features 216A for image A and point features 216B for image B).

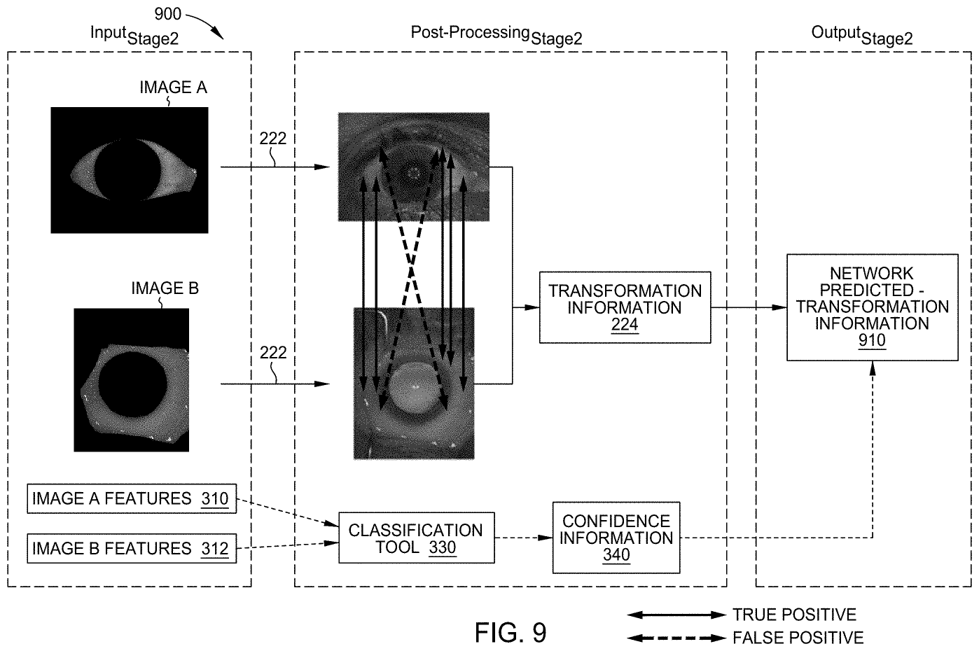

Referring back to FIG. 5, at block 508, the registration component generates a set of transformation information (e.g., transformation information 224), based at least in part on an evaluation of the point feature(s) and the eye regions within the set of images. In certain embodiments, the registration component can generate the set of transformation information by evaluating the masks and point features using one or more computer vision algorithms (e.g., computer vision algorithms 222). By way of example, FIG. 9 illustrates a second stage 900 of ophthalmic image registration in which transformation information is generated using one or more computer vision algorithms, according to certain embodiments. As shown, during a post-processing stage of the second stage 900, the point features 216 of the input images A and B are matched using at least one computer vision algorithm (e.g., RANSAC, SIFT, NMS, etc.) and the set of transformation information 224 is generated.

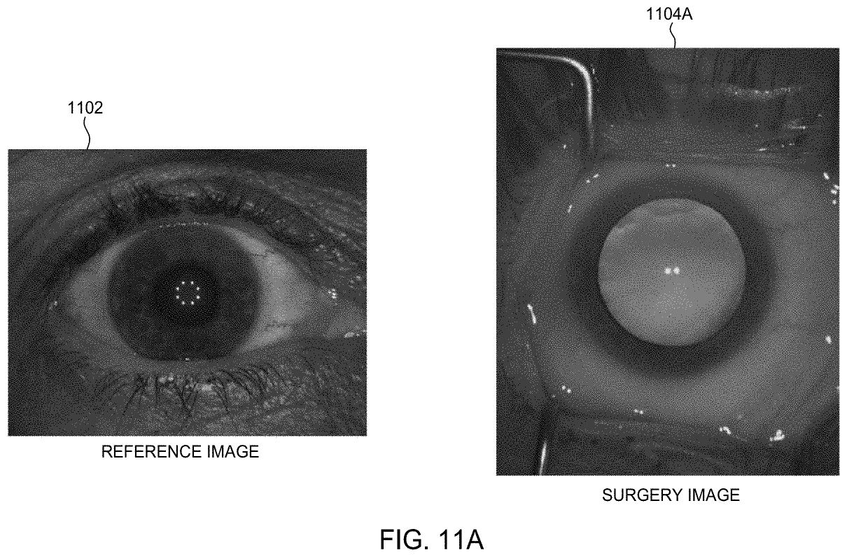

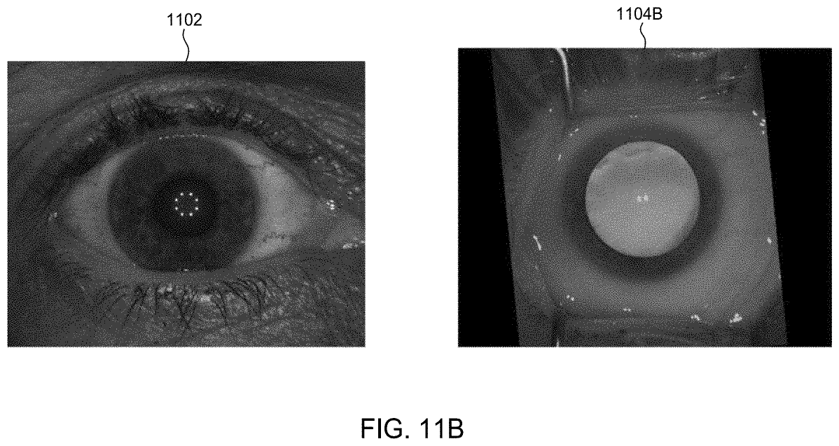

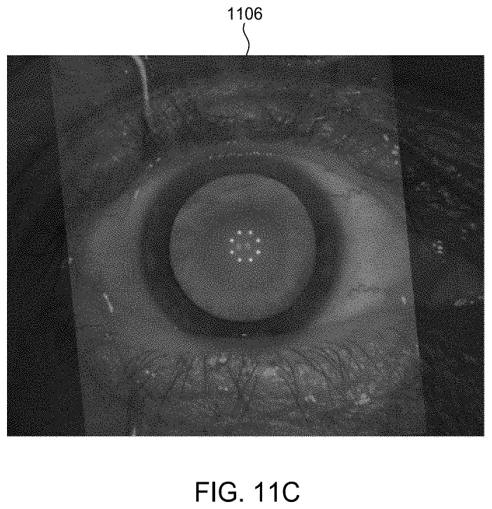

At block 510, the registration component transforms at least one of the set of images based on the set of transformation information. For example, the transformation information can include scaling information, translation information, and/or rotation information for transforming at least one of the set of images, such that the point features in each image are aligned (or are in the same coordinate system). By way of example, FIG. 11B illustrates a transformed surgery image 1104B that may be output from the registration component, given the reference image 1102 and the surgery image 1104A illustrated in FIG. 11A as inputs to the registration component.

In certain embodiments, the registration component can generate an image overlay with the transformed set of images (e.g., in the same coordinate system). By way of example, FIG. 11C illustrates an image overlay 1106 that may be generated by the registration component, based on the transformed surgery image 1104B and the reference image 1102.

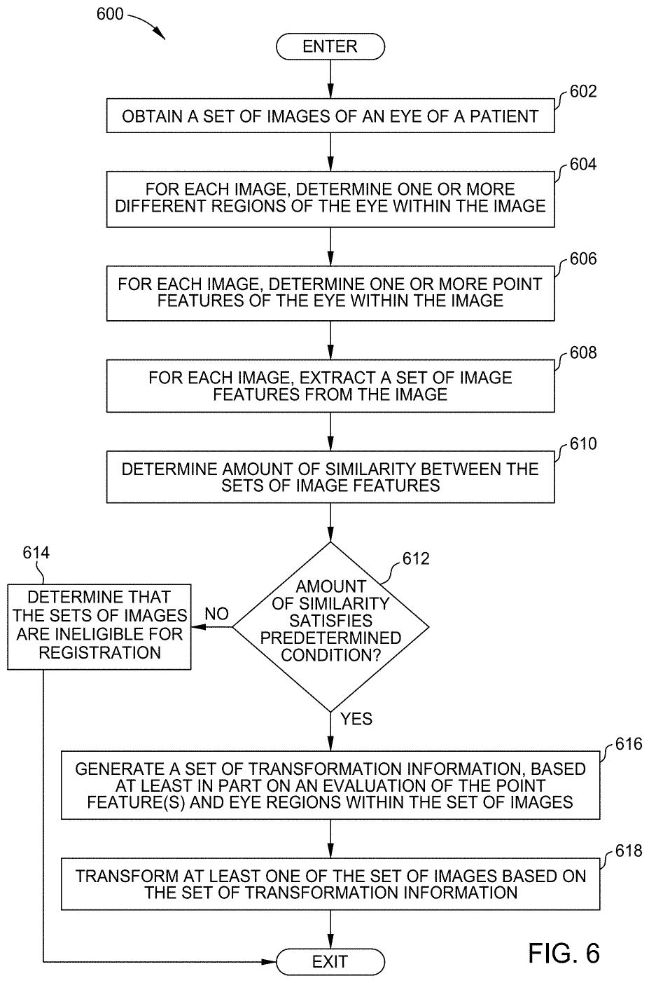

FIG. 6 is a flowchart of another method 600 for performing ophthalmic image registration, according to certain embodiments. The method 600 may be performed by a registration component (e.g., registration component 152).

Method 600 enters at block 602, where the registration component obtains a set of images of an eye of a patient (e.g., patient 102). Each image of the set of images may be captured at a different time, at a different angle/position, using a different sensor/modality, etc.

At block 604, the registration component, for each image, determines one or more different regions of the eye (e.g., masks(s) 214) within the image. For example, the registration component can perform a segmentation operation on each image using one or more neural networks (e.g., neural networks 212) to identify regions of the skin, sclera, iris, pupil, etc.

At block 606, the registration component, for each image, determines one or more point features (e.g., point features 216) of the eye within the image. The one or more point features may correspond to one or more unique landmarks or points of the eye. The registration component can generate a set of point features based on evaluating each image using the one or more neural networks (e.g., neural networks 212).

At block 608, the registration component, for each image, extracts a set of image features from the image. In certain embodiments, the set of image features may be extracted from a middle layer of the one or more neural networks. For example, assuming the U-net architecture 400 is used as the neural network, the middle layer may correspond to feature map 412 in step i of the U-net architecture 400.

In certain embodiments, the operations in block 604, block 606, and block 608 may be performed using a single neural network 212. By way of example, FIG. 7 illustrates a first stage 700 of ophthalmic image registration using a single neural network 212, according to certain embodiments. Here, the single neural network 212 receives the input images A and B and outputs masks 214 and point features 216 for each input image A, image A features 310, and image B features 312.

In certain embodiments, the operations in block 604, block 606, and block 608 may be performed using multiple neural networks 212. By way of example, FIG. 8 illustrates a first stage 800 of ophthalmic image registration using multiple neural networks 212-1 and 212-2, according to certain embodiments. In the first stage 800, the neural network 212-1 can segment different regions of each input image A and B. The neural network 212-2 receives the mask (or segment) information output from the neural network 212-1 and generates, for each image, a set of point features within the image (e.g., point features 216A for image A and point features 216B for image B), image A features 310, and image B features 312.

Referring back to FIG. 6, at block 610, the registration component determines an amount of similarity between the sets of image features. For example, the registration component can perform a cross-correlation (or another type of comparison) of the sets of image features to determine an amount of correlation (or similarity) between the sets of image features. By way of example, in the second stage 900 illustrated in FIG. 9, during the post-processing stage, the classification tool 330 can determine an amount of correlation between the image A features 310 and image B features 312 and generate confidence information 340 that includes an indication of the amount of similarity between the sets of image features.

At block 612, the registration component determines whether the amount of similarity satisfies a predetermined condition. The predetermined condition may include the amount of similarity being greater than a threshold. If the amount of similarity does not satisfy the predetermined condition, then the registration component determines that the set of images are ineligible for registration (block 614).

On the other hand, if the amount of similarity does satisfy the predetermined condition, then the registration component generates a set of transformation information, based at least in part on an evaluation of the point feature(s) and eye regions within the set of images (e.g., using one or more computer vision algorithms 222) (block 616). By way of example, in the second stage 900 illustrated in FIG. 9, registration component may output a network-predicted transformation information 910, which includes an indication of the confidence information 340 and transformation information 224. At block 618, the registration component transforms at least one of the set of images based on the set of transformation information.

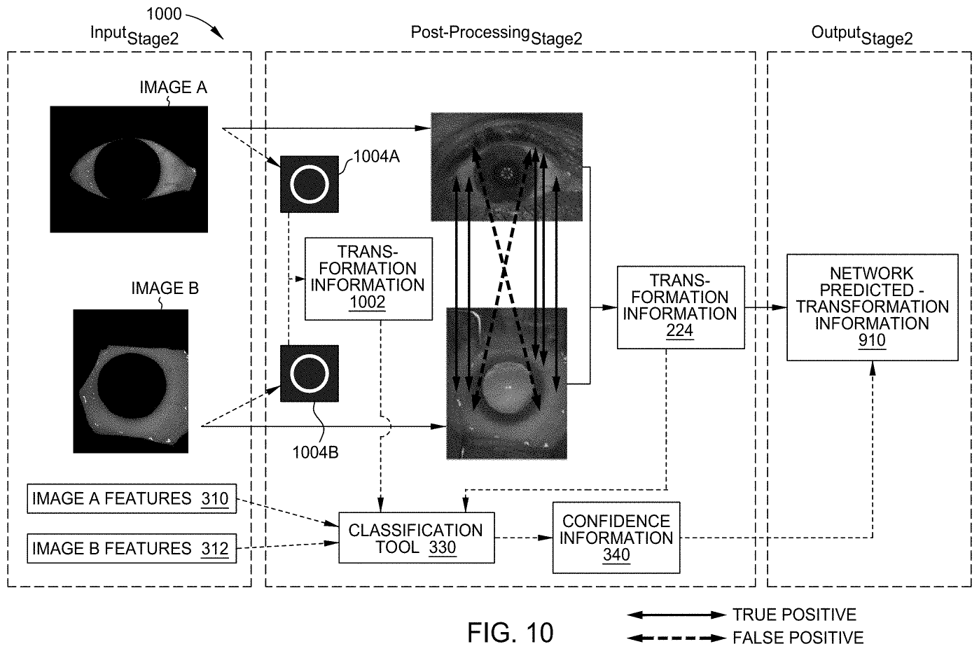

In certain embodiments, in addition to determining the amount of similarity between the sets of image features, the registration component may also determine whether the input images are registerable using information obtained from one or more computer vision algorithms. By way of example, FIG. 10 illustrates an example second stage 1000 of ophthalmic image registration, according to certain embodiments. During the post-processing stage of the second stage 1000, the point features of the input images A and B are matched using a first one or more computer vision algorithms 222 (e.g., RANSAC/SIFT) in order to generate the transformation information 224. Additionally, the input images A and B may be evaluated with a second one or more computer vision algorithms 222 in order to generate the transformation information 1002. In certain embodiments, the second one or more computer vision algorithms 222 may include a thresholding algorithm. The registration component can perform thresholding on the input images A and B to estimate the limbus center and radius of the eye within each image (shown as images “1004A” and “1004B” in FIG. 10). The transformation information 1002 can be generated based on the comparison of the limbus center and radius information from input image A to the limbus center and radius information from input image B. For example, the transformation information 1002 can include scaling information and/or translation information for transforming at least one of the set of images.

Continuing with FIG. 10, the classification tool 330 receives the image A features 310, the image B features 312, the transformation information 1002, and the transformation information 224 and generates the confidence information 340, based on an evaluation of the input information. The confidence information 340 can indicate whether the input images A and B are eligible for registration. For example, the confidence information 340 can include an indication of the amount of correlation between the features of the input images. In certain embodiments, the confidence information 340 can indicate that the input images are not eligible for registration upon determining at least one of the following: (i) the transformation information 1002 is inconsistent with the transformation information 224 or (ii) the amount of correlation between the image A features 310 and the image B features 312 is less than a threshold. The transformation information 1002 may be inconsistent with the transformation information 224, for example, when the scaling information/translation information in the transformation information 1002 does not match the scaling information/translation information in the transformation information 224.

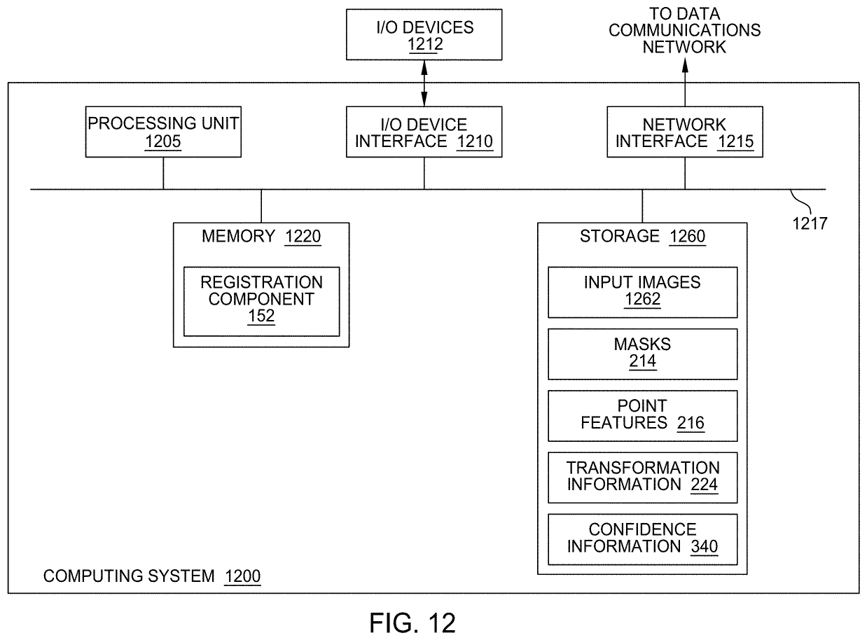

FIG. 12 illustrates an example computing system 1200 configured to perform ophthalmic image registration, according to certain embodiments. As shown, the computing system 1200 includes, without limitation, a processing unit 1205, a network interface 1215, a memory 1220, and storage 1260, each connected to a bus 1217. The computing system 1200 may also include an I/O device interface 1210 connecting I/O devices 1212 (e.g., keyboard, display and mouse devices) to the computing system 1200. The computing system 1200 is generally under the control of an operating system (not shown). Examples of operating systems include the UNIX operating system, versions of the Microsoft Windows operating system, and distributions of the Linux operating system. (UNIX is a registered trademark of The Open Group in the United States and other countries. Microsoft and Windows are trademarks of Microsoft Corporation in the United States, other countries, or both. Linux is a registered trademark of Linus Torvalds in the United States, other countries, or both.) More generally, any operating system supporting the functions disclosed herein may be used.

The processing unit 1205 can include one or more central processing units (CPUs) and/or one or more graphics processing units (GPUs). The processing unit 1205 retrieves and executes programming instructions stored in the memory 1220 as well as stored in the storage 1260. The bus 1217 is used to transmit programming instructions and application data between the processing unit 1205, I/O device interface 1210, storage 1260, network interface 1215, and memory 1220. Note, processing unit 1205 is included to be representative of a single CPU, multiple CPUs, a single CPU having multiple processing cores, a single GPU, multiple GPUs, a single GPU having multiple processing cores, or any combination thereof. The memory 1220 is generally included to be representative of a random access memory. The storage 1260 may be a disk drive or flash storage device. Although shown as a single unit, the storage 1260 may be a combination of fixed and/or removable storage devices, such as fixed disc drives, removable memory cards, optical storage, network attached storage (NAS), or a storage area-network (SAN). Illustratively, the memory 1220 includes the registration component 152, which is discussed in greater detail above. Further, storage 1260 includes masks 214, point features 216, transformation information 224, and confidence information 340, which are also discussed in greater detail above. Storage 1260 also includes input images 1262, which can include any set of input images obtained for an ophthalmic image registration procedure.

In summary, embodiments of the present disclosure enable the performance of ophthalmic image registration using interpretable deep learning based AI. The systems and/or methods described herein are particularly advantageous for providing additional data points and information to improve diagnostic accuracy and/or the effectiveness of surgical procedures.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a c c, b-b, b-b-b, b-b-c, c-c, and c-c-c or any other ordering of a, b, and c).

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

Within a claim, reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.” The word “exemplary” is used herein to mean “serving as an example, instance, or illustration.” Any aspect described herein as “exemplary” is not necessarily to be construed as preferred or advantageous over other aspects.

What is claimed is:

1. An ophthalmic system for performing ophthalmic image registration, comprising:
one or more ophthalmic imaging devices configured to generate a plurality of images of an eye of a user, each of the plurality of images comprising a different view of the eye;
a memory comprising executable instructions; and
a processor in data communication with the memory and configured to execute the executable instructions to:
determine, within each image of the plurality of images, one or more segmented regions of the eye within the image, based on evaluating the image with one or more neural networks;
determine, within each image of the plurality of images, a set of point features of the eye within the one or more segmented regions of the eye, based on evaluating the image with at least one of the one or more neural networks;
generate a set of transformation information for transforming at least one of the plurality of images, based on performing one or more image processing operations on the set of point features within each image of the plurality of images; and
transform the at least one of the plurality of images, based on the set of transformation information, wherein the processor being configured to transform the at least one of the plurality of images comprises at least one of scaling the at least one of the plurality of images, translating the at least one of the plurality of images, or rotating the at least one of the plurality of images, such that the plurality of images with the different views are in a same coordinate system.

2. The ophthalmic system of claim 1, wherein the processor is further configured to execute the executable instructions to extract, from at least one of the one or more neural networks, a set of features associated with each of the plurality of images.

3. The ophthalmic system of claim 2, wherein the set of features associated with each of the plurality of images is extracted from a middle layer of the at least one of the one or more neural networks.

4. The ophthalmic system of claim 3, wherein the processor is further configured to execute the executable instructions to:
determine an amount of similarity between the plurality of images, based on comparing the sets of features; and
provide an indication of the amount of similarity.

5. The ophthalmic system of claim 4, wherein the at least one of the plurality of images is transformed upon determining that the amount of similarity satisfies a predetermined condition.

6. The ophthalmic system of claim 1, wherein the one or more neural networks is a single neural network.

7. The ophthalmic system of claim 1, wherein the one or more neural networks comprises (i) a first neural network configured to provide an indication of the one or more segmented regions of the eye and (ii) a second neural network configured to provide an indication of the set of point features.

8. The ophthalmic system of claim 1, wherein the processor is further configured to execute the executable instructions to generate an image overlay comprising the at least one transformed image of the plurality of images and one or more non-transformed images of the plurality of images.

9. A computer-implemented method for performing ophthalmic image registration, the computer-implemented method comprising:
obtaining a plurality of images of an eye of a user, each of the plurality of images comprising a different view of the eye;
determining, within each image of the plurality of images, one or more segmented regions of the eye within the image, based on evaluating the image with one or more neural networks;
determining, within each image of the plurality of images, a set of point features of the eye within the one or more segmented regions of the eye, based on evaluating the image with at least one of the one or more neural networks;
generating a set of transformation information for transforming at least one of the plurality of images, based on performing one or more image processing operations on the set of point features within each image of the plurality of images; and
transforming the at least one of the plurality of images, based on the set of transformation information, wherein transforming the at least one of the plurality of images comprises at least one of scaling the at least one of the plurality of images, translating the at least one of the plurality of images, or rotating the at least one of the plurality of images, such that the plurality of images with the different views are in a same coordinate system.

10. The computer-implemented method of claim 9, further comprising extracting, from at least one of the one or more neural networks, a set of features associated with each of the plurality of images.

11. The computer-implemented method of claim 10, wherein the set of features associated with each of the plurality of images is extracted from a middle layer of the at least one of the one or more neural networks.

12. The computer-implemented method of claim 11, further comprising:
determining an amount of similarity between the plurality of images, based on comparing the sets of features; and
providing an indication of the amount of similarity.

13. The computer-implemented method of claim 12, wherein the at least one of the plurality of images is transformed upon determining that the amount of similarity satisfies a predetermined condition.

14. The computer-implemented method of claim 9, wherein the one or more neural networks is a single neural network.

15. The computer-implemented method of claim 9, wherein the one or more neural networks comprises (i) a first neural network configured to provide an indication of the one or more segmented regions of the eye and (ii) a second neural network configured to provide an indication of the set of point features.

16. The computer-implemented method of claim 9, wherein each of the one or more neural networks is based on a U-net architecture.

17. The computer-implemented method of claim 9, further comprising generating an image overlay comprising the at least one transformed image of the plurality of images and one or more non-transformed images of the plurality of images.

18. The computer-implemented method of claim 9, wherein the plurality of images comprises (i) a first image of the eye of the user captured during a pre-operative period and (ii) a second image of the eye of the user captured during an intra-operative period.

19. The computer-implemented method of claim 9, wherein the plurality of images comprises (i) a first set of images captured with a first imaging sensor of a stereo camera and (ii) a second set of images captured with a second imaging sensor of the stereo camera.

20. The computer-implemented method of claim 9, wherein each of the plurality of images was at least one of: (i) captured at a different time instance; (ii) capturing using a different modality; or (iii) captured at a different angle.
